(19) United States
(12) Patent Application Publication
(10) Pub. No.: US 2021/0063334 A1
(43) Pub. Date: Mar. 4, 2021

(54) APPARATUS AND METHODS FOR DETECTION OF DIABETES-ASSOCIATED MOLECULES USING ELECTROCHEMICAL IMPEDANCE SPECTROSCOPY

(71) Applicants: Arizona Board of Regents on behalf of Arizona State University, Scottsdale, AZ (US); Mayo Foundation for Medical Education and Research, Rochester, MN (US)

(72) Inventors: Jeffrey LaBelle, Tempe, AZ (US); Chi Lin, Van Nuys, CA (US); David Probst, Chandler, AZ (US); Koji Sode, Chapel Hill, NC (US); Curtiss Cook, Scottsdale, AZ (US)

(21) Appl. No.: 16/981,157

(22) PCT Filed: Mar. 18, 2019
(86) PCT No.: PCT/US19/22703

§ 371 Date: Sep. 15, 2020

Related U.S. Application Data
(60) Provisional application No. 62/644,167, filed on Mar. 16, 2018.

(51) Int. Cl. G01N 27/02; G01N 27/327; G01N 33/543

(52) U.S. Cl. CPC G01N 27/026; G01N 33/543; G01N 27/3276

(57) ABSTRACT

Methods and apparatus for detecting binding of a diabetes-related target molecule analyte in a sample utilizing Electrochemical Impedance Spectroscopy (EIS). Sensor electrodes include a diabetes-related target-capturing molecule immobilized thereto, and an EIS-based imaginary impedance measurement is utilized to arrive at a concentration of the analyte.

### BACKGROUND

Diabetes Mellitus (DM) encompasses a series of chronic metabolic diseases characterized by inadequate glucose metabolism¹. It is quickly becoming a worldwide epidemic, involving nearly 24 million people in the United States, and costing nearly 250 billion dollars². According to the American Diabetes Association, by the year 2034 the number of diagnosed and undiagnosed people with diabetes will increase from 23.7 million to 44.1 million³. With such an increase in prevalence, there has also been a large need for next generation technology to help manage the disease with better portability and increased sensitivity⁴. Currently, diabetes management involves monitoring glucose levels daily, either discretely or continuously, and glycated hemoglobin (HbA1c) levels periodically^{5,6}.

### SUMMARY

Embodiments disclosed herein relate to a rapid and label-free insulin biosensor with high sensitivity and accuracy. In certain embodiments, an insulin biosensor prototype capable of detecting insulin in a physiological range without complex data normalization is disclosed.

Further embodiments relate to electrochemical impedance spectroscopy use to identify an optimal frequency specific to insulin detection on a gold disk electrode with insulin antibody immobilized, which can be accomplished by conjugating the primary amines of an insulin antibody to the carboxylic bond of the self-assembling monolayer on the gold surface.

Other embodiments relate to the use of imaginary impedance to detect insulin concentration and to establishment of an optimal frequency of insulin at 810.5 Hz, which is characterized by having the highest sensitivity and sufficient specificity.

These and other aspects are further described in the following figures and detailed description of certain embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 depicts a representation of certain selective markers (molecules) that influence glucose management.

FIG. 2 shows, in accordance with certain embodiments, an exemplary sensor fabrication process and detection mechanism. A: Bare GDE. B: 1 mM 16-MHDA self-assembled linker. C: EDC/NHS coupling. D: immobilization of 156 μM insulin antibody. E: 1% ethanolamine blocking; addition of an aptamer between and below the antibodies for detection of glucose (such that more than one diabetes-related molecule can be detected at the same time). F: Binding of insulin antigen to antibody. G: Electrochemical cell, which in certain embodiments comprises a 1000 μL pipet tip with counter and reference electrodes.

FIG. 3 is, in accordance with certain embodiments, an example of the quality control (QC) mentioned above, and shows the average peak location, and magnitude of the desired electrodes within the test data. B) This figure shows the logarithmic fit (slope) and RSQ values by fitting the imaginary impedance against target insulin concentrations across the frequency sweep. 810.5 Hz was found to be the optimal binding frequency (OBF) at which both slope and RSQ peaked.

FIG. 4 is, in accordance with certain embodiments, a representation of a calibration curve of 0, 50, 100, 200, 250, 500, 750, 1000, 1500 ρM based off imaginary impedance readings of insulin detected with N=7 repetitions at each concentration. Error bars were calculated from the standard deviations.

FIG. 5 is, in accordance with certain embodiments, an image of the circuit used to model the electrochemical cell. Rₛₒₗ is the resistance due to solution, Rₑₜ is the electron transfer resistance. Q is used to represent the constant phase element (CPE) or the imperfect capacitor of the system.

FIG. 6 is, in accordance with certain embodiments, a calibration curve relating the calculated charge transfer resistance against the change in concentration of insulin in ρM.

### DETAILED DESCRIPTION OF CERTAIN ILLUSTRATIVE EMBODIMENTS

Embodiments herein relate to apparatus and methods for detecting one or more analytes in a bodily fluid sample utilizing Electrochemical Impedance Spectroscopy (EIS). In particular embodiments, an electrochemical sensor is configured to (e.g., operably configured to) provide an electrochemical impedance measurement of an analyte and includes a diabetes-related target-capturing molecule immobilized (e.g., through a chemical linker) to a working electrode.

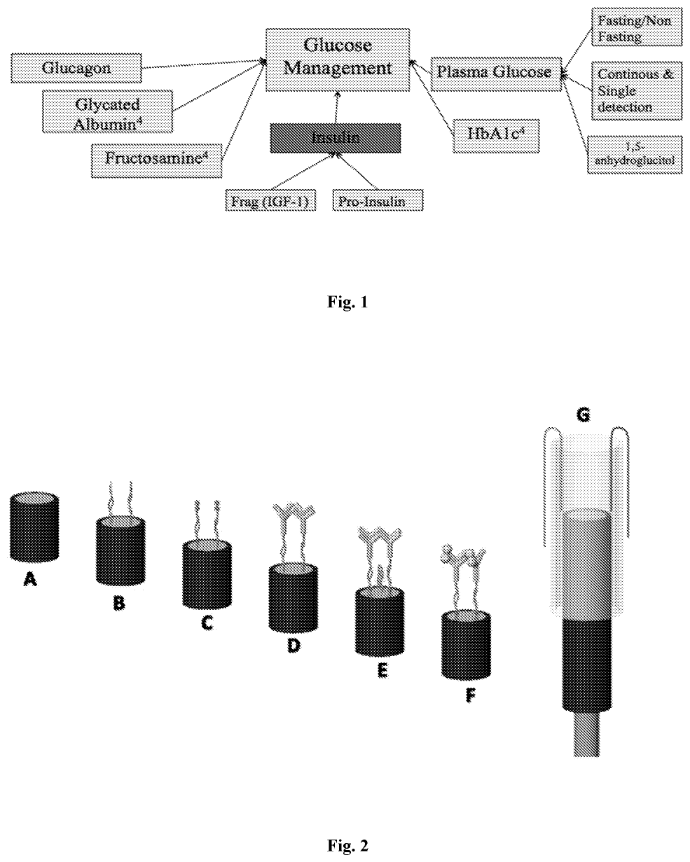

Presently, detection and monitoring of metabolic status in DM is achieved through detection of a single molecular marker: glucose. Other molecular markers, such as insulin, are important but currently not easily measured at the point of care. The development of multi-marker detection assays is desirable; generally speaking, many studies have shown that monitoring multiple biomarkers associated with a complex disease can enhance the accuracy of disease diagnosis, prognosis, management, and treatment⁷⁻¹⁰. FIG. 1 shows key biomarkers involved in glucose management⁵ and measuring of these biomarkers can give a better understanding of a patient's state of health. However, exactly how to go about accurate multi-marker detection is difficult and has yet to be accomplished to any practical degree in the context of insulin

Among the many diabetes-related target molecules in FIG. 1, insulin is a biomarker that directly affects glucose levels in achieving glucose homeostasis¹¹⁻¹³. The current state of the art for insulin detection are enzyme-linked immunosorbent assay (ELISA) and High Performance Liquid Chromatography (HPLC). While these techniques are specific and sensitive, they require specialized laboratory technicians and time consuming procedures^{14,15}. There is a need for a simple, label free, and rapid insulin sensor suitable for a point-of-care setting in addition to a glucose sensor.

The momentum for developing electrochemical insulin sensors has been increasing in the past few years¹⁶⁻¹⁹. The inventors recently have showed that, using the imaginary impedance of EIS, a biomarker will have an optimal binding frequency (OBF) at which the change in imaginary impedance best correlates to the change in target concentrations²⁰. The inventors have already characterized glucose previously using EIS and have shown its feasibility in glucose detection¹⁵. Additional biomarkers can be explored to build a multi-marker sensing platform monitoring all the major biomarkers of DM, providing the most accurate information for medical intervention and glycemic control.

In some embodiments, the devices and methods include a diabetes-related target-capturing molecule, such as an antibody, an aptamer, or other molecule recognized in view of the teachings in this application by those of ordinary skill in the art as suitable for their specific applications. Aptamers are single-stranded, synthetic oligonucleotides that fold into 3-dimensional shapes capable of binding non-covalently with high affinity and specificity to a target molecule. The diabetes-related target-capturing molecules may capture targets such as insulin, glucose, or other diabetes-related molecules. Moreover, the detection of binding of such molecules may be continuous in certain sensor embodiments.

In some embodiments, multiple targets are captured and imaginary impedance measurements are taken at distinct frequencies to then determine the binding (and related concentration) of each target.

### NON-LIMITING EXAMPLES

### Reagents and Chemicals

All chemical reagents were purchased from Sigma (St Louis, Mo., USA) unless stated otherwise. The 10 mM phosphate buffer saline (PBS) tablets were purchased from Calbiochem (Gibbstown, N.J., USA), potassium hexacyanoferrate (III) from EMD Chemicals (Billerica, Mass., USA), and sulfo-derivative of N-hydroxysuccinimide sodium salt (NETS) from Toronto Research Chemicals (Toronto, Ontario, Canada). The redox probe reagent used was 100 mM potassium ferricyanide dissolved in pH 7.4 PBS.

### Sensor Fabrication and Testing

In this non-limiting example, in accordance with certain embodiments, the sensor includes 3 electrodes: working gold disk electrodes (GDEs), reference silver/silver chloride electrodes, and counter platinum electrodes acquired from CH Instruments (Austin, Tex., USA). All EIS measurements were performed at room temperature using a CHI660C Electrochemical Analyzer from CH Instrument at the electrode's formal potential from 1 Hz to 100 kHz. A Buehler felt pad with 0.05 μg grit aluminum oxide particles was used to polish the GDEs with 10 figure-eight motions, followed by a 20-minute sonication in deionized water. After electrode polishing, cyclic voltammetry (CV) from −1.0 V to 1.0 V was used to obtain the formal potential and bare electrode EIS was performed to evaluate sensor cleanliness.

Once the sensors were cleaned, the SAM (self-assembled monolayer) was created by incubating 1 mM of 16-mercaptohexadecanoic acid (MHDA) for one hour at room temperature. The sensors were then rinsed and stored dry overnight to ensure proper deposition of SAM, as SAMs takes hours to reach their final thickness and contact angles^{21,22}. The carboxylate groups of the 16-MHDA were activated by incubating the sensor in 10 mM 1-ethyl-3-(3-dimethylaminopropyl) carbodiimide (EDC) and 80 mM sulfo-NHS for one hour at room temperature. After rinsing with DI, 100 μL of 156 μM of insulin antibody prepared in pH 7.4 PBS was incubated onto the electrode surface for one hour. After rinsing with PBS the sensors were blocked with 1% ethanolamine for 30 minutes to block any remaining active sites, completing the sensor fabrication process. The schematic of sensor preparation can be found in FIG. 2. The finished sensors were stored at 277.15 K until testing. All sensors were brought to room temperature before running each test. Insulin antigen gradients were prepared through serial dilution with PBS. Each sample contained 200 mM potassium ferricyanide and equal volume of insulin antigen to form 100 μL total solution volume. Final insulin samples were made according to the physiological concentration range from 0 ρM to 1500 ρM to establish a calibration curve.

### Quality Control

Electrodes were prepared in batches of eighteen and all electrodes were analyzed using Electrochemical Impedance Spectroscopy. After measuring the post-MHDA impedance, the quality control (QC) was executed by selecting only the electrodes with similar peak frequencies and impedance magnitudes that are within 6% to 10% relative standard deviation (% RSD). Only the QC-passing sensors would then proceed with immobilization.

### Determination of OBF and Circuit Modeling

Once EIS was performed, the imaginary impedance values were correlated to target concentrations to calculate slope and R-square values (RSQ) across the frequency sweep. The OBF is the frequency at which the slope peaks with RSQ values above 0.85. All circuit modeling was performed using ZsimpWin software.

### Results

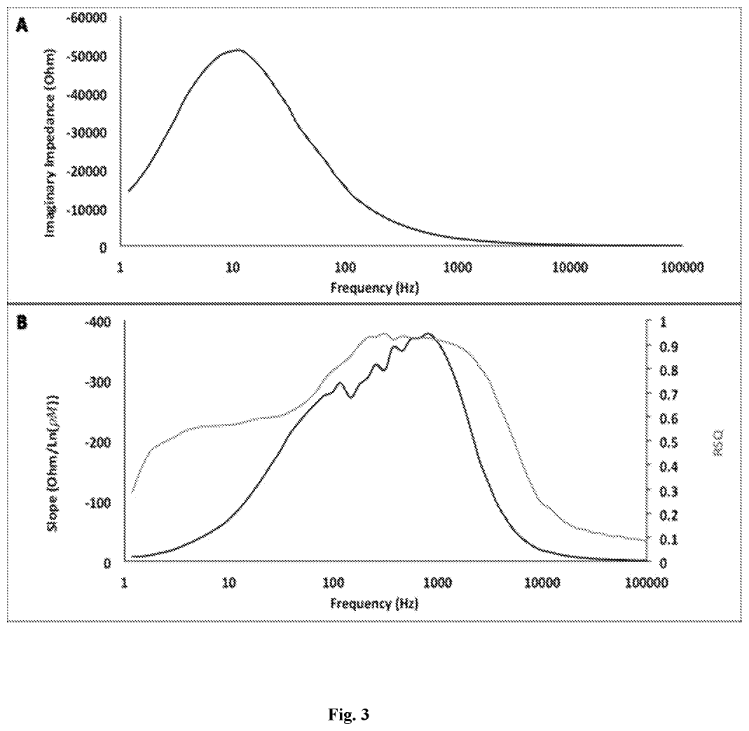

Using the illustrative methods described above, the impedance responses from 7 electrodes were used to determine the OBF of insulin, which was found to be 810.5 Hz (FIG. 3). This relationship was evident in all 7 electrodes as the peak frequency shifts consistently comparing to the post-MHDA results (FIG. 3a).

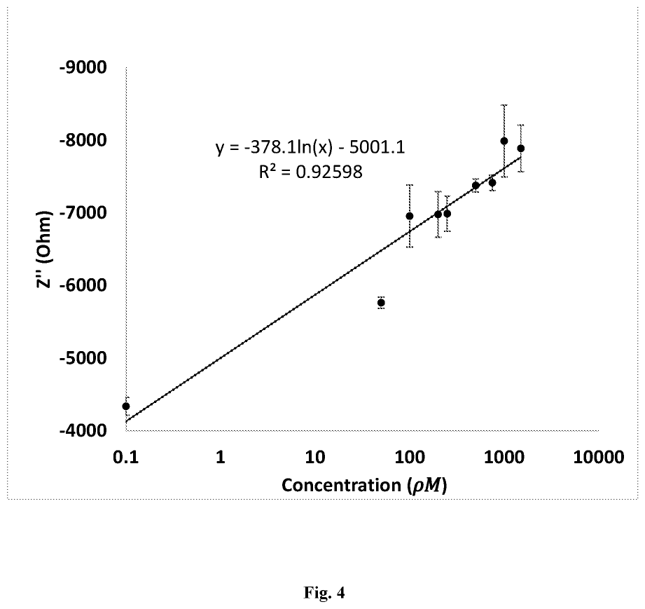

FIG. 4 shows the relationship between the imaginary impedance and the target insulin concentration range (0 ρM to 1500 ρM) at the OBF of 810.5 Hz. The target insulin range is the physiological insulin range²³. The correlations between the impedance and concentrations were 0.926 and the logarithmic slope was −378.1 ln(x) with x being the concentration of insulin and the intercept being −5001.1. The slope is represented as negative due to the nature of imaginary impedance values however; the graph represents correlation between increasing concentration and impedance. The % RSDs for this physiological concentration range from low to high concentrations were 11%, 5%, 26%, 19%, 14%, 5%, 5%, 25%, and 16%. The lower limit of detection was calculated to be 2.64 ρM.

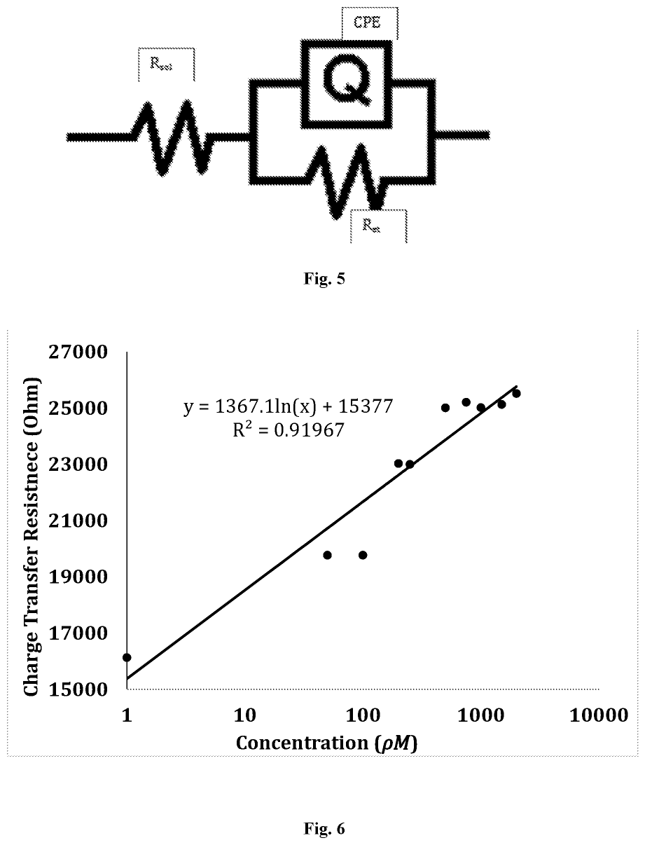

Using ZsimpWin, one example of a benefit circuit model that well-describes the electrochemical system of insulin sensor can be obtained (FIG. 5). The solution resistance and the electron transfer resistance were both modeled as resistors and were labeled as Rₛₒₗ and Rₑₜ, respectively. The pseudo-capacitor is modeled as Q and represents the piece of the system that can be correlated to the molecular recognition element being used.

FIG. 6 shows the correlation between charge transfer resistance and target insulin concentrations derived from equivalent circuit modeling, a standard method of analyzing EIS data²⁴.

### Electrochemical Impedance Results

Comparing the results between FIGS. 3a and 3b, it was evident that the shift in peak frequency is due to the binding of insulin antibody. The shifts are reproducible as the QC was executed rigorously. Since the slope peaks at 810.5 Hz with RSQ value of 0.93, 810.5 Hz is determined to be the OBF of insulin. However, it is important to note that there is often a trade-off between the sensitivity (slope) and specificity (RSQ) when considering the optimal frequency of EIS¹⁵. FIG. 4 shows the calibration curve for purified insulin at 810.5 Hz. For insulin, a logarithmic fit with slope of −378.1 Ohm/Ln(ρM) and RSQ of 0.93 was found to correlate imaginary impedance with concentration of insulin. The purpose for running a calibration curve experiment is because a hand held or other device is programmed with these equations, and, upon running EIS on an unknown sample, the calibration curve would convert an imaginary impedance reading into an insulin concentration.

In other words, after obtaining the impedance reading from an unknown sample analyte, the number can be plugged back into the calibration curve to obtain the concentration of the unknown sample analyte. For an example involving insulin, a −7500 ohm reading at 810.5 Hz would result in 1000 pM of insulin according to FIG. 4.

The lower limit of detection (LLD) and dynamic range are important parameters in determining the efficiency of the system. The LLD and dynamic range were calculated based off the standard deviation and slope of the system. The LLD was found to be 2.64 ρM and dynamic range from 50 pM to 1500 pM, which meets clinical needs. From a clinical standard detection of insulin, ELISA can accurately detect labeled insulin at 1.39 ρM²⁵. This is slightly lower then what the inventors have demonstrated with this sensor prototype, but with optimization of the electrode design, the LLD may be lowered to that of ELISA. Even more so, techniques such as ELISA or high-performance liquid chromatography have labeling steps and many associated techniques that can be performed only in laboratories. EIS on the other hand, is a label free technique, and the sensor prototype can be translated into screen printed sensors, allowing the possibility of point-of-care detection with a portable device and disposable test strips similar to the setup of self-monitoring of blood glucose devices^{14,26}.

The Food and Drug Administration requires all glucose meters to be within 20% variance from standards²⁷. Currently, the replicated results show that across all sample concentrations the % RSDs ranges from 5% to 26%, suggesting there are still room for improvements. Although batch analysis has helped eliminate some of the variance between GDEs, polishing and reusing GDEs is a significant source of variance as surface roughness of gold can affect SAM formation²⁸, affecting the capacitance of imperfect parallel plate capacitor (IPPC) explained in later section. Transition to screen printed sensors will reduce the variance of surface roughness under consistent manufacturing procedures and rigorous quality control.

The inventors have shown that the EIS method of using imaginary impedance can very well detect insulin in the physiological range. Within certain embodiments, even smaller concentration interval sizes may be employed (such as about 1 pM), which is equivalent to a gold standard ELISA to distinguish between even the smallest changes in concentration. In certain embodiments, the technologies described herein may be embodied within a point-of-care (POC) device. Notably, unlike other publications on insulin detection there was no modification to the insulin solution via pH^{17,18}.

### Circuit Analysis

Generally, EIS is analyzed with equivalent circuit modeling. Typically, the best-fit circuit for a semi-circle looking Nyquist plot is the Randles circuit, which models the electrochemical interactions as a resistance-capacitor circuit in parallel. The electron transfer resistance can be used to derive a calibration curve linking back to input concentration^{25,29}. However, recently some researchers have demonstrated the use of a modified Randles circuit that implements a constant phase element (CPE) to model the capacitance^{20,23,30}. CPE is commonly referred to as either a leaky or imperfect parallel plate capacitor (IPPC).

The bottom plate is the surface of electrode and the top plate is the top of the SAM with Molecular Recognition Elements (MREs) immobilized owing to SAM's insulating property³¹. The MREs different shape, orientation and size alter the smoothness of SAM in various ways, constituting the IPPC. As binding occurs, the target-MRE complex further alters the capacitance of the IPPC, affecting the electron transferring properties and impedance signals, which is evident in FIG. 6. This model gives a better description of the actual system when compared to the ideal Randles.

Since imaginary impedance correlates to capacitance²⁴, the inventors used imaginary impedance to correlate target concentration to reflect the impedance signal generated from changes in CPE, which the inventors believe to have less noise than using the complex impedance approach and omits the trouble of circuit modeling. Owing to this nature, it's no surprise that the LLD in imaginary impedance (2.64 ρM) is lower than that of the complex impedance approach (14.46 ρM).

An insulin biosensor prototype POC device has been developed. Detection of insulin and other molecules affecting individuals with diabetes will greatly enhance the ability of individuals with diabetes to better control their own blood glucose levels. With a reproducible LLD of 2.26 ρM the example embodiment herein suggests that imaginary impedance based techniques are not only sensitive enough to detect physiological concentrations in purified solution of small proteins such as insulin but can also compete with current SOTA devices as well.

In certain embodiments, the biosensor described herein is embodied in a disposable strip that is capable of insulin detection in clinical samples. In certain embodiments, screen printed electrodes (SPEs) may be created using a MPM Accuflex Speedline screen printer. Depending on the embodiment and specific dimension of the sensor, machine overhead, and the amount of sensors fabricated, the current cost of a sensor can be as low as 1$ per sensor with order size of 45,000 sensors. In certain embodiments, the insulin sensor may be translated onto such SPEs.

Additionally, examples of a dual-marker detection sensor using the imaginary impedance of EIS would detect glucose and insulin simultaneously at their respective optimal binding frequencies (OBFs). For example, if the OBF of glucose is 31.5 Hz and insulin's is 810.5 Hz, the impedance reading at 31.5 Hz can be correlated to the glucose's concentration and the impedance reading at 810.5 Hz will be the concentration of insulin. In certain embodiments, the impedance reading correlated to glucose concentration may be the impedance reading at a frequency within a range of 25 Hz to 35 Hz, 28 Hz to 32 Hz, or 30 Hz to 32 Hz, or at frequency of about 31.5 Hz. In certain embodiments, the impedance reading correlated to insulin concentration may be the impedance reading at a frequency within a range of 775 Hz to 825 Hz, 790 Hz to 820 Hz, 800 Hz to 820 Hz, 808 Hz to 812 Hz, or 810 Hz to 811 Hz, or at a frequency of about 810.5 Hz. As those of ordinary skill in the art will appreciate in view of the teachings in this application, the impedance reading may be correlated to a concentration of a different diabetes-related analyte at a frequency other than those set forth in the nonlimiting examples herein (e.g., an analyte other than insulin or glucose at a frequency at or approximating the OBF for that particular diabetes-related analyte).

While certain embodiments have been shown and described herein, it will be obvious to those skilled in the art that such embodiments are provided by way of example only. Numerous variations, changes, and substitutions will now occur to those skilled in the art without departing from the disclosure herein. It should be understood that various alternatives to the embodiments described herein may be employed. It is intended that the following claims define the scope of the methods and structures described and their equivalents be covered thereby.

### REFERENCES

1. Sudharsan B, Peeples M, Shomali M. Hypoglycemia prediction using machine learning models for patients with type 2 diabetes. J Diabetes Sci Technol. 2015; 9(1):86-90.
2. Olokoba A B, Obateru O A, Olokoba L B. Type 2 Diabetes Mellitus: A Review of Current Trends. Oman Med J. 2012 Jul. 16; 27(4):269-273.
3. Huang E S, Basu A, O'Grady M, Capretta J C. Projecting the future diabetes population size and related costs for the U S. Diabetes Care. 2009; 32(12):2225-2229.
4. Turner A P. Biosensors—sense and sensitivity. Science. 2000; 290(5495):1315-1317.
5. Juraschek S P, Steffes M W, Selvin E. Associations of alternative markers of glycemia with hemoglobin A1c and fasting glucose. Clin Chem. 2012; 58(12):1648-1655.
6. Larsen M L, Høder M, Mogensen E F. Effect of long-term monitoring of glycosylated hemoglobin levels in insulin-dependent diabetes mellitus. N Engl J Med. 1990; 323(15):1021-1025.
7. Boer R A, Lok D J, Jaarsma T, van der Meer P, Voors A A, Hillege H L, van Veldhuisen D J. Predictive value of plasma galectin-3 levels in heart failure with reduced and preserved ejection fraction. Ann Med. 2011; 43(1):60-68.
8. Sullivan S D, Garrison Jr L P, Rinde H, Kolberg J, Moler E J. Cost-effectiveness of risk stratification for preventing type 2 diabetes using a multi-marker diabetes risk score. J Med Econ. 2011; 14(5):609-616.
9. Wang T J, Gona P, Larson M G, Levy D, Benjamin E J, Tofler G H, Jacques P F, Meigs J B, Rifai N, Selhub J. Multiple biomarkers and the risk of incident hypertension. Hypertension. 2007; 49(3):432-438.
10. Wang T J, Gona P, Larson M G, Tofler G H, Levy D, Newton-Cheh C, Jacques P F, Rifai N, Selhub J, Robins S J. Multiple biomarkers for the prediction of first major cardiovascular events and death. N Engl J Med. 2006; 355(25):2631-2639.
11. Rosen E D, Spiegelman B M. Adipocytes as regulators of energy balance and glucose homeostasis. Nature. 2006; 444(7121):847-853.
12. Rodbard D. Evaluating Quality of Glycemic Control Graphical Displays of Hypo- and Hyperglycemia, Time in Target Range, and Mean Glucose. J Diabetes Sci Technol. 2015; 9(1):56-62.
13. Kilpatrick E S, Maylor P W, Keevil B G. Biological variation of glycated hemoglobin: implications for diabetes screening and monitoring. Diabetes Care. 1998; 21(2):261-264.
14. Adamson T L, Cook C B, LaBelle J T. Detection of 1,5-Anhydroglucitol by Electrochemical Impedance Spectroscopy. J Diabetes Sci Technol. 2014 Mar. 1; 8(2):350-355.
15. Adamson T L, Eusebio F A, Cook C B, LaBelle J T. The promise of electrochemical impedance spectroscopy as novel technology for the management of patients with diabetes mellitus. Analyst. 2012; 137(18):4179-4187.
16. Gerasimov J Y, Schaefer C S, Yang W, Grout R L, Lai R Y. Development of an electrochemical insulin sensor based on the insulin-linked polymorphicregion. Biosens Bioelectron. 2013; 42:62-68.
17. Xu M, Luo X, Davis J J. The label free picomolar detection of insulin in blood serum. Biosens Bioelectron. 2013 January; 39(1):21-25.
18. Luo X, Xu M, Freeman C, James T, Davis J J. Ultrasensitive label free electrical detection of insulin in neat blood serum. Anal Chem. 2013; 85(8):4129-4134.
19. Martinez-Periñán E, Revenga-Parra M, Gennari M, Pariente F, Mas-Ballesté R, Zamora F, Lorenzo E. Insulin sensor based on nanoparticle-decorated multiwalled carbon nanotubes modified electrodes. Sens Actuators B Chem. 2016; 222:331-338.
20. Lin C, Ryder L, Probst D, Caplan M, Spano M, LaBelle J. Feasibility in the development of a multi-marker detection platform. Biosens Bioelectron. 2016;
21. Ulman A. Formation and structure of self-assembled monolayers. Chem Rev. 1996; 96(4):1533-1554.
22. Pan Y, Sonn G A, Sin M L, Mach K E, Shih M-C, Gau V, Wong P K, Liao J C. Electrochemical immunosensor detection of urinary lactoferrin in clinical samples for urinary tract infection diagnosis. Biosens Bioelectron. 2010; 26(2):649-654.
23. Lu Y, Li H, Zhuang S, Zhang D, Zhang Q, Zhou J, Dong S, Liu Q, Wang P. Olfactory biosensor using odorant-binding proteins from honeybee: Ligands of floral odors and pheromones detection by electrochemical impedance. Sens Actuators B Chem. 2014 March; 193:420-427.
24. Barsoukov E, Macdonald J R. Impedance spectroscopy: theory, experiment, and applications. John Wiley & Sons; 2005.
25. Kuznetsov B, Shumakovich G, Koroleva O, Yaropolov A. On applicability of laccase as label in the mediated and mediatorless electroimmunoassay: effect of distance on the direct electron transfer between laccase and electrode. Biosens Bioelectron. 2001; 16(1):73-84.
26. Malkoc A, Sanchez E, Caplan M R, La Belle J T. Electrochemical-Nucleic Acid Detection with Enhanced Specificity and Sensitivity. J Biosens Bioelectron. 2015; 6(2):1.
27. Freckmann G, Schmid C, Baumstark A, Pleus S, Link M, Haug C. System accuracy evaluation of 43 blood glucose monitoring systems for self-monitoring of blood glucose according to DIN E N ISO 15197. J Diabetes Sci Technol. 2012; 6(5):1060-1075.
28. Hajisalem G, Min Q, Gelfand R, Gordon R. Effect of surface roughness on self-assembled monolayer plasmonic ruler in nonlocal regime. Opt Express. 2014; 22(8):9604-9610.
29. La Belle J T, Fairchild A, Demirok U K, Verma A. Method for fabrication and verification of conjugated nanoparticle-antibody tuning elements for multiplexed electrochemical biosensors. Methods. 2013; 61(1):39-51.
30. Lu Y, Zhang D, Zhang Q, Huang Y, Luo S, Yao Y, Li S, Liu Q. Impedance spectroscopy analysis of human odorant binding proteins immobilized on nanopore arrays for biochemical detection. Biosens Bioelectron. 2016; 79:251-257.
31. Boubour E, Lennox R B. Insulating properties of self-assembled monolayers monitored by impedance spectroscopy. Langmuir. 2000; 16(9):4222-4228.

1. An apparatus for detecting one or more diabetes-related analytes in a bodily fluid sample utilizing Electrochemical Impedance Spectroscopy (EIS), comprising:
an electrochemical sensor operably configured to provide an electrochemical impedance measurement of a diabetes-related analyte in the fluid, the sensor including a diabetes-related target-capturing molecule immobilized to a working electrode.

2. The apparatus of claim 1, wherein the target-capturing molecule is an antibody.

3. The apparatus of claim 2, wherein the antibody is against insulin.

4. The apparatus of claim 1, wherein the working electrode includes a self-assembled monolayer coupled to the target-capturing molecule.

5. (canceled)

6. The apparatus of claim 1, wherein the target-capturing molecule is an aptamer.

7. (canceled)

8. The apparatus of claim 6, wherein the aptamer is configured to bind glucose.

9. The apparatus of claim 6, wherein the aptamer is configured to bind insulin.

10. The apparatus of claim 1, wherein the electrochemical sensor is operably configured to provide an imaginary impedance measurement.

11. (canceled)

12. An apparatus for detecting insulin in a bodily fluid sample utilizing Electrochemical Impedance Spectroscopy (EIS), comprising:
an electrochemical sensor operably configured to provide an electrochemical impedance measurement of insulin in the fluid, the sensor including an insulin-capturing molecule immobilized to a working electrode.

13. The apparatus of claim 12, wherein the insulin-capturing molecule is an antibody.

14. The apparatus of claim 12, wherein the insulin-capturing molecule is an aptamer.

15. (canceled)

16. (canceled)

17. (canceled)

18. A method for detecting binding of a diabetes-related target molecule analyte in a sample utilizing Electrochemical Impedance Spectroscopy (EIS), comprising:
contacting an electrode with the sample, wherein the electrode includes a diabetes-related target-capturing molecule immobilized thereto, and wherein the electrode is operably configured to provide an EIS-based imaginary impedance measurement of the sample; and
detecting the binding of the diabetes-related target molecule analyte in the sample to the target-capturing molecule by detecting a change in an imaginary impedance measurement.

19. The method of claim 18, further including establishing an optimal frequency for the diabetes-related target molecule analyte.

20. The method of claim 18, wherein the target-capturing molecule is an antibody.

21. The method of claim 20, wherein the antibody is against insulin.

22. The method of claim 18, wherein the electrode includes a self-assembled monolayer coupled to the target-capturing molecule.

23. (canceled)

24. The method of claim 18, wherein the target-capturing molecule is an aptamer.

25. (canceled)

26. The method of claim 24, wherein the aptamer is configured to bind glucose.

27. The method of claim 24, wherein the aptamer is configured to bind insulin.

28. The method of claim 18, wherein the electrode is operably configured to detect the binding of more than one analyte, wherein binding is detected at a distinct frequency for each analyte.

29. (canceled)
